(19) United States
(12) Patent Application Publication
(10) Pub. No.: US 2018/0036338 A1
(43) Pub. Date: Feb. 8, 2018

(54) FLOWABLE HEMOSTATIC COMPOSITION

(71) Applicants: BAXTER INTERNATIONAL INC., Deerfield, IL (US); BAXTER HEALTHCARE SA, Glattpark (Opfikon) (CH)

(72) Inventors: Paul Jefferey Sanders, Greendale, WI (US); Timothy Michael Fulghum, Lakemoor, IL (US); John Joseph Anthony Barry, Northbrook, IL (US)

(21) Appl. No.: 15/537,765

(22) PCT Filed: Dec. 18, 2015
(86) PCT No.: PCT/US2015/066767

§ 371 Date: Jun. 19, 2017

Related U.S. Application Data
(60) Provisional application No. 62/094,578, filed on Dec. 19, 2014.

(51) Int. Cl. A61K 31/738; A61K 38/48; A61L 26/00

(52) U.S. Cl. CPC A61K 31/738; A61L 26/008; A61K 38/4833; C12Y 304/21005; A61L 26/0023; A61L 26/0066; A61L 2400/04; A61L 2400/06

(57) ABSTRACT

The present disclosure relates, in general, to hemostatic compositions comprising polysaccharide based polymers, such as amylopectins, amylose, dextrins, maltodextrins and icodextrin and modified forms thereof, cross-linked with a cross-linking agent that are useful as a hemostatic composition. Also provided are methods of treating injury and slowing or stopping bleeding using a hemostatic composition disclosed herein.

### FIELD OF THE DISCLOSURE

The present disclosure, relates, in general to a hemostatic composition comprising a polysaccharide- or starch-based polymer, including amylopectin, amylose, dextrins, such as maltodextrin, and modified forms thereof, and use in stopping or reducing bleeding in a subject.

### BACKGROUND

Hemostatic agents and sealants are currently used as an aid to stop bleeding, including hemorrhaging, during surgery. The FDA has approved hemostatic matrices, such as FLOSEAL® (Baxter International), for use in patients to augment the natural clotting cascade or to mechanically stop bleeding at a surgical or wound site. FLOSEAL® is a hemostatic sealant comprising human thrombin within a gelatin matrix.

TACHOSIL® (Baxter International) is a collagen sponge matrix combined with the coagulation factors fibrinogen and thrombin. It is activated by blood and body fluids to form a clot that glues the sponge to the tissue surface. Hemostasis and stoppage of bleeding is reached within a few minutes.

Other forms of hemostatic compositions include a hemostatic sponge, pad or powder comprising plant based material such as purified plant starch linked to a carboxymethyl group to modify the starch (Starch Medical). See also U.S. Pat. No. 8,623,842, which discloses a hemostatic composition comprising gelatinized potato starch.

The present disclosure provides an improved hemostatic composition.

### SUMMARY OF THE DISCLOSURE

The present disclosure provides a hemostatic composition comprising a polysaccharide or starch, such as a cross-linked amylopectin, maltodextrin or cross-linked modified amylopectin maltodextrin, in a flowable hydrogel that can be used as a hemostatic matrix. Currently, many hemostatic compositions used in patients are comprised of animal derived starting material (e.g., gelatin) to manufacture a hemostatic matrix. The present composition use non-animal, plant based polysaccharide/starch polymers to make the hemostatic matrix.

In various embodiments, the disclosure provides a hemostatic composition comprising a cross-linked polysaccharide or starch, such as amylopectin, amylose, a dextrin, such as maltodextrin or icodextrin, or cross-linked modified forms thereof. In various embodiments, the disclosure provides a hemostatic composition comprising a cross-linked amylopectin, icodextrin or maltodextrin or a cross-linked modified amylopectin, icodextrin or maltodextrin.

In various embodiments, the hemostatic composition is a flowable hydrogel. In various embodiments, the hemostatic composition is in a powdered form, a sprayable form, or is used in a sponge, pad, film or fiber.

In various embodiments, the hemostatic composition comprises a crosslinking agent selected from the group consisting of epichlorohydrin, bis-epoxypropylether, ethylene glycol-bis-epoxy propyl ether, sodium trimetaphosphate (STMP), adipic-acetic anhydride, phosphorous oxychloride, a diepoxide, vinylcyclohexene dioxide, butadiene dioxide, formaldehyde, glutaraldehyde and genipin.

In various embodiments, the hemostatic composition further comprises one or more procoagulants. Exemplary procoagulants include, but are not limited to, clotting factors, such as thrombin, fibrin, Factor VIII (FVIII), Factor VII (FVII), Factor IX (FIX), von Willebrand Factor (vWF), Factor II (FII), Factor V (FV), Factor X (FX), Factor XI (FXI), Factor XII (FXII), and Factor XIII (FXIII), tissue factor, collagen; styptics including anti-hemorrhagic agents and astringents; and other hemostatic agents. In one embodiment, the hemostatic composition further comprises thrombin.

In various embodiments, the hemostatic composition further comprises a functional group selected from the group consisting of an amine, an aldehyde, a phosphate, a phosphonate, a sulfate, a sulfonate, and a carboxylate group.

In various embodiments, the hemostatic composition activates the coagulation cascade or enhances binding to tissues.

In various embodiments, the amylopectin, amylose or dextrin composition is between 2000 to 500,000 Da, between 10,000 to 300,000 Da, between 20,000 to 250,000 Da between 50,000 to 200,000 Da, between 75,000 to 150,000 Da, between 10,000 to 100,000, between 25,000 to 75,000 or between 40,000 to 60,000 Da.

In various embodiments, the maltodextrin is selected from the group consisting of low molecular weight, medium molecular weight or high molecular weight maltodextrin. In one embodiment, the low molecular weight maltodextrin is less than 2,000 Da. In one embodiment, the medium molecular weight maltodextrin is between 2,000 and 45,000 Da. In one embodiment, the high molecular weight maltodextrin is greater than 45,000 Da. In various embodiments, the icodextrin is between 13000-19000 Da.

In various embodiments, the hemostatic composition further comprises one or more of an active agent and/or an additive. An active agent contemplated herein refers to an agent that provides pharmacological activity or other direct effect in the diagnosis, cure, mitigation, treatment, or prevention of disease, or affects the structure or any function of the body of man or animals. An additive contemplated herein refers to an agent that acts to improve or facilitate activity of an active ingredient or stability or another property of the hemostatic composition. In various embodiments, the active agent or additive is selected from the group consisting of a polysaccharide, polyvinylalchohol, polyethyleneimine, chitosan, alginic acid, a fucoidan, hyaluronic acid, heparin, heparan, chondroitin sulfate, amylopectin, amylose, xanthan gum, guar gum, carageenans, carboxymethyl cellulose, carboxymethyl maltodextrin, carboxymethyl icodextrin, carboxymethyl starch, agarose, and natural or synthetic polymers (e.g., neutral, cationic, anionic, basic, or acidic polymers).

In various embodiments, the polysaccharide or starch polymer, including amylopectin, amylose, a dextrin, icodextrin or maltodextrin, is derived from plant material, bacteria, fungi or algae. In various embodiments, the plant material is selected from the group consisting of corn, soy, rice, tapioca, wheat, waxy corn, or waxy rice.

In various embodiments, when the hemostatic composition is in a powder form, the composition is in particle form, wherein the particle is between from 10 to 1000 μm, from 50 to 800 μm, from 50 to 700 μm, from 150 to 700 μm, from 200 to 700 μm, from 300 to 550 μm, and from 350 to 550 μm.

In various embodiments, the flowable form of the hemostatic composition contains particles of which more than 50% (w/w) have a size of 100 to 1000 μm, or particles of which more than 80% (w/w) have a size of 100 to 1000 μm. It is contemplated that the flowable form contains crosslinked biocompatible polymer in an amount of 5 to 30% (w/w), of 10 to 25% (w/w), or of 12 to 20% (w/w).

In various embodiments, the hemostatic matrix has a degradation time of greater than 3 hours, 4 hours, 5 hours, 6 hours, 12 hours, 24 hours, 2 days, 3 days, 5 days, 10 days, 15 days, 20 days, 25 days, or 30 days.

In various embodiments, the hemostatic matrix has an E Swell within the range of from 400% to 1300%, from 500% to 1100%, or from 600% to 900%,

In various embodiments, the hemostatic matrix has an Q Swell that approaches the E swell. It is contemplated that in certain embodiments, a high Q swell may be beneficial, and one that approaches the E swell may reach maximum swelling more rapidly.

Other parameters to measure stability and efficacy of the hemostatic composition include assays for thrombin time (TT), prothrombin time (PT), activated partial thromboplatin time (APTT), fibrinogen (FIB) platelet factor 4 (PF4); anticoagulation parameters including antithrombin III (AT-III); fibrinolytic parameters including plasminogen (PLG), fibrin degradation product (FDP) and D-dimer; blood viscosity (BV) and plasma viscosity (PV).

In certain embodiments, the composition is stable when pre-packaged in a syringe or other device for delivery to a patient. In various embodiments, the composition is stable for 1, 2, 3, 4, 5, 6, 78, 9 or 10 days inside or outside the delivery device. In certain embodiments, the composition is stable at room temperature for 1, 2, 3, 4, 5, 6, 7, 8, 9, 10, 11, 12, 13, 14, 15, 16, 17, 18, 19, 20, 21, 22, 23 or 24 hours in flowable form.

In various embodiments, the disclosure provides a method of treating an injury selected from the group consisting of a wound, a hemorrhage, damaged tissue and bleeding tissue comprising administering a hemostatic composition described herein to the site of injury.

Also contemplated is a method for slowing or stopping bleeding comprising applying to a bleed area a hemostatic composition described herein.

In various embodiments, the injury or bleeding occurs during surgery.

In various embodiments, the hemostatic composition is applied as a hydrogel, in powder form, or as a pad, a film or a fiber. In various embodiments, the hemostatic composition is in powdered form and the composition is first mixed with liquid to form a flowable composition. In various embodiments, the liquid is selected from the group consisting of saline, water, buffer solutions, and protein solutions. It is contemplated that the protein solutions comprises one or more clotting factors or natural or synthetic procoagulants.

In various embodiments, the hemostatic composition used in the method stops bleeding within seconds or minutes. It is contemplated that the hemostatic composition stops or reduces bleeding in a subject within 10, 20, 30, 40, 50, or 60 seconds, or within 1, 2, 3, 4, 5, 6, 7, 8, 9, 10 or 15 minutes.

Also contemplated is a kit for the treatment of an injury selected from the group consisting of a wound, a hemorrhage, damaged tissue and bleeding tissue or for slowing or stopping bleeding comprising a) a hemostatic composition described herein and b) instructions for use.

In various embodiments of the kit, the hemostatic composition is in dry form, and the kit further comprises a pharmaceutically acceptable liquid or diluent for reconstitution of the hemostatic composition.

Also contemplated are methods of making a hemostatic composition as described herein. In various embodiments, the disclosure provides a process for making a hemostatic composition comprising cross-linking polymeric compositions (polysaccharide, natural or synthetic, or non-carbohydrate based polymers, natural or synthetic) amenable to modification with a bifunctional crosslinker selected from the group consisting of epichlorohydrin, bis-epoxypropylether, ethylene glycol-bis-epoxy propyl ether, sodium trimetaphosphate (STMP), adipic-acetic anhydride, phosphorous oxychloride, formaldehyde, a diepoxide, vinylcyclohexene dioxide, butadiene dioxide, formaldehyde, gluaraldehyde and genipin by contacting the polymer with the crosslinker in aqueous or organic or combinations thereof solutions, such as a buffer or protein solution in varying molar equivalents (polymer:crosslinker) ranging from 0.01:1 to 20:1, under varying pH conditions ranging from 7.0 to 14.0, for period of at least 1 to 24 hours at 0-100° C., such as 4° C., 20° C., 25° C., 37° C., 42° C. or 50° C., and isolating the cross-linked polymer composition.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE FIGURES

FIG. 1 depicts the Visual Bleeding Scale Used to Assess Bleeding Pre- and Post-Sealant Application.

FIG. 2 is a graph showing Hemostatic Success versus Time of FLOSEAL VH S/D and Flowable Maltodextrin.

FIG. 3 shows Hemostatic Success (All-time points/Lesions) of FLOSEAL VH S/D and Flowable Maltodextrin.

### DETAILED DESCRIPTION

The present disclosure provides a hemostatic composition comprising a polysaccharide or starch-based polymer in which the properties of the polymer have been altered to achieve an end-product that has an optimized hemostatic profile. For example, the polymer is crosslinked to alter the degradation timing and the swelling/fluid uptake of the particles and porosity of the particles, for use as a hemostatic matrix. The polysaccharide-based hydrogels can be utilized in a hydrated/flowable format or utilized as dry powders.

Unless otherwise defined, all technical and scientific terms used herein have the same meaning as commonly understood by one of ordinary skill in the art to which the present disclosure belongs. The following references provide one of skill with a general definition of many of the terms used in this disclosure: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

As used in the present disclosure and the appended claims, the terms “a”, “an” and “the” include plural reference as well as singular reference unless the context clearly dictates otherwise.

As used herein, the following terms have the meanings ascribed to them unless specified otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5% or 0.1% of a given value or range. Whenever the term “about” or “approximately” precedes the first numerical value in a series of two or more numerical values, it is understood that the term “about” or “approximately” applies to each one of the numerical values in that series.

A “hemostatic composition” refers to a composition useful to stop or reduce bleeding that results from injury or surgery. It is contemplated that a hemostatic composition of the present disclosure comprises a polysaccharide or starch-based polymer, such as amylopectin, amylose or a dextrin, including amylodextrin, cyclodextrin, maltodextrin, icodextrin, or modified variants thereof.

A “flowable” composition or “hydrogel” refers to a substantially liquid, slightly viscous solution, solid, semi-solid solid, pseudoplastic, or plastic structure containing an aqueous component to produce a gelatinous or jelly-like mass, or paste-like solution that has the properties of being able to flow through a syringe or other device and be administering to a subject. The flowable hydrogel is a liquid-like, slightly viscous solution, or paste-like solution at room temperature and body temperature. In various embodiments, a flowable composition is one that holds shape when extruded through a syringe or other device for administering to a subject.

A “subject” of diagnosis or treatment is a human or non-human animal, including a mammal or a primate. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “effective amount” of a therapeutic means a dosage sufficient to produce a desired result on a health condition, pathology, or disease of a subject or for a diagnostic purpose. The desired result may comprise a subjective or objective improvement in the recipient of the dosage. “Therapeutically effective amount” refers to that amount of an agent effective to produce the intended beneficial effect on health.

The term “hemostatic agent” as used herein refers to compositions that are used to slow or stop bleeding in a subject and/or can promote the coagulation cascade to stop bleeding. Glick et al, (Indian Dermatol Online J. 4(3):172-176, 2013) and Sundaram et al., (Indian J Urol. 26(3):374-378, 2010) describe various hemostatic agents. Exemplary hemostatic agents include, but are not limited to, aluminum chloride, ferric sulfate (Monsel's solution) silver nitrate, zinc chloride paste, thrombin, gelatins (e.g,. porcine gelatin and gelatin mixtures), microporous polysaccharide spheres, hydrophilic polymers with potassium salts (HPPS), oxidized regenerated cellulose, microfibrillar collagens, aminocaproic acid, aprotinin, tranexamic acid, alginate, mineral zeolite, cyanoacrylates, chitosan and kaolin.

Hemostatic Compositions

Hemostatic compositions have been prepared using various polymers, and different compositions in the field of surgery and wound care. For example, Genipin-crosslinked biological material, including genipin-crosslinked gelatin, has been disclosed previously (see, e.g., U.S. Pat. No. 6,608,040, EP2181722, WO2008/076407, Bigi et al., Biomaterials 23 (2002), 4827-4832; Yao et al., Mat. Chem. Phys. 83 (2004), 204-208; Turo et al., Int. J. Biol. Macromol. (2011), doi: 10.1016; Chiono et al., J. Materl. Sci.: Mater Med. (2008) 19:889-898). U.S. Patent Publication 20130129710 discloses a flowable hemostatic composition which uses a genipin-type crosslinker in the hemostatic materials.

Atyabin et al. (Archives of Pharmacal Research 29: 1179-1186, 2006) discloses cross-linked starch microspheres. Satyanarayana et al., (Superdisintegrants, Int. J. Chem Tech Res 3:1786-1798, 2010) describes synthesis and evaluation of 2-Hydroxymethylene cross-linked dextrins. U.S. Pat. No. 6,419,957, describes cross-linked high amylose starch having functional groups as a matrix for the slow release of pharmaceutical agents. See also U.S. Patent Publication 20100255101, U.S. 2007/0087061, U.S. Pat. No. 6,060,461, WO 2008/117300, WO 2009/091549, and WO 2009153798.

The present disclosure contemplates use of a polysaccharide or starch-based polymer, such as amylopectin, amylose or a dextrin, including amylodextrin, cyclodextrin, maltodextrin, icodextrin, or derivatives and fragments thereof in a hemostatic composition. Amylopectin is a soluble polysaccharide, highly branched polymer of glucose, found in plants and, along with amylose, is a component of starch. Starch is made of about 70% amylopectin by weight. The glucose units of amylopectin are linked in a linear way with α(1→4) glycosidic bonds. Branching takes place with α(1→6) bonds occurring every 24 to 30 glucose units. Amylose is insoluble in water. Amylose comprises α(1→4) bound glucose molecules but contains very few α(1→6) bonds.

Dextrins are a group of low-molecular-weight carbohydrates produced by the hydrolysis of starch. Maltodextrin is a shortchain starch sugar. Cyclodextrins are cyclical destrins formed by degradation of starch by certain bacteria. Amylodextrin is a linear dextrin or short chained amylose that can be produced by enzymatic hydrolysis of the alpha-1,6 glycosidic bonds or debranching amylopectin.

Contemplated herein are cross-linking agents, including, but not limited to epichlorohydrin, bis-epoxypropylether, ethylene glycol-bis-epoxy propyl ether, sodium trimetaphosphate (STMP), adipic-acetic anhydride, phosphorous oxychloride, formaldehyde, diepoxides, vinylcyclohexene dioxide or butadiene dioxide (as described in Patent WO 2009153798 A1), formaldehyde/gluaraldehyde and genipin. A study by Atyabi et al. demonstrated that starch particle size, swelling ratio and release of small molecules contained within the particle could be controlled by changing the type and concentration of cross-linking agent as well as cross-linking time.

In certain embodiments, prior to or after cross-linking the polysaccharide polymer, including amylopectin, maltodextrin or icodextrin, can be further functionalized to contain groups such as amines, aldehyde, phosphate, phosphonate, sulfate, sulfonate, carboxylate, which can interact with tissues and bodily fluids with intent of activating the coagulation cascade and/or enhance binding to tissues. These added properties (e.g. anionic or cationic charge) could provide hemostatic properties to the hydrogel and thus eliminate the need for hemostatic agents such as thrombin. The powdered or flowable hemostatic compositions can be formulated with or without thrombin.

In various embodiments, the cross-linked polysaccharide polymer is dried. In a dried state, the hemostatic composition is storage-stable for long time even at elevated temperatures (e.g. more than 20° C., more than 30° C. or even more than 40° C.). Dryness conditions include cross-linked polysaccharide polymers, including, icodextrin or maltodextrin, which are dried to have a moisture content of below 15% (w/w), below 10%, below 5%, or below 1%. For a dried composition, it is contemplated that the composition has a re-hydration rate of at least 2 g/g, of at least 3.5 g/g, or of 3.75 g/g or higher. Re-hydration rates of similar powders prepared without the re-hydration aids are typically below three, and a percentage increase in re-hydration rate will usually be at least 5%, at least 10%, or at least 25% or higher.

In another embodiment the product is supplied in a hydrated or wet state where the hydrating solution may be a biocompatible buffer or solution.

In one embodiment, the hemostatic composition according to the present disclosure contains the cross-linked polysaccharide polymer in particulate form, for example as granular material. The granular material can rapidly swell when exposed to a fluid (i.e. the pharmaceutically acceptable diluent) and in this swollen form is capable of contributing to a flowable paste that can be applied to a bleeding site. The polysaccharide polymer, e.g. maltodextrin or icodextrin, may be provided as a film which can then be milled to form a granular material. Most of the particles contained in this granular material (e.g. more than 90% w/w) have particle sizes of from 10 to 1000 μm, from 50 to 800 μm, from 50 to 700 μm, from 150 to 700 μm, from 200 to 700 μm, from 300 to 550 μm, and from 350 to 550 μm.

In various embodiment, the hemostatic composition is liquid absorbing. For example, upon contact with liquids, e.g. aqueous solutions or suspensions (especially a buffer or blood) the polymer takes up the liquid and will display a degree of swelling, depending on the extent of hydration. The material absorbs from at least 300%, about 400% to about 2000%, from about 500% to about 1300% water or aqueous buffer by weight, corresponding to a nominal increase in diameter or width of an individual particle of subunit in the range from e.g. approximately 50% to approximately 500%, or from approximately 50% to approximately 250%. For example, if the (dry) granular particles have a preferred size range of 0.01 mm to 1.5 mm, or of 0.05 mm to 1 mm, the fully hydrated composition (e.g., after administration on a wound or after contact with an aqueous buffer solution) may have a size range of 0.05 mm to 3 mm, especially of 0.25 mm to 1.5 mm.

The equilibrium swell of the hemostatic composition of the present disclosure may generally range, e.g., from 400% to 1300%, from 500% to 1100%, or from 600% to 900%, depending on its intended use. Such equilibrium swell may be controlled, e.g., (for a crosslinked polymer) by varying the degree of cross-linking, which in turn is achieved by varying the cross-linking conditions, such as the duration of exposure of a cross-linking agent, concentration of a cross-linking agent, cross-linking temperature, and the like. Materials having differing equilibrium swell values perform differently in different applications. The ability to control crosslinking and equilibrium swell allows the compositions of the present invention to be optimized for a variety of uses. In addition to equilibrium swell, it is also important to control the hydration of the material immediately prior to delivery to a target site. Hydration and equilibrium swell are, of course, intimately connected. A material with 0% hydration is non-swollen. A material with 100% hydration will be at its equilibrium water content. Hydrations between 0% and 100% will correspond to swelling between the minimum and maximum amounts.

For finishing the crosslinked polymer to a pharmaceutically acceptable hemostatic composition a pharmaceutically acceptable diluent is used.

In various embodiments, the pharmaceutically acceptable diluent is an aqueous solution and may contain a substance selected from the group consisting of NaCl, CaCl₂, sodium acetate, sodium lactate, sodium citrate, sodium caprate and mannitol. For example, a pharmaceutically acceptable diluent comprises water for injection, and—independently of each other—50 to 200 mM NaCl (e.g., 150 mM), 10 to 80 mM CaCl₂ (e.g., 40 mM), 1 to 50 mM sodium acetate (e.g., 20 mM) and up to 10% w/w mannitol (e.g., 2% w/w). In various embodiments, the diluent can also include a buffer or buffer system so as to buffer the pH of the reconstituted dry composition, e.g., at a pH of 3.0 to 10.0, at a pH of 6.4 to 7.5, or at a pH of 6.9 to 7.1.

In certain embodiments, the pharmaceutically acceptable diluent comprises thrombin, from 10 to 1000 I.U. thrombin/ml, or from 250 to 700 I.U. thrombin/ml. In various embodiments, the hemostatic composition in ready to use form contains 10 to 100,000 International Units (I.U.) of thrombin, from 100 to 10,000 I.U., or from 500 to 5,000 I.U. The thrombin concentration in the ready-to-use composition is in the range of 10 to 10,000 I.U., or from 50 to 5,000 I.U.; or from 100 to 1,000 I.U./ml. The diluent is used in an amount to achieve the desired end-concentration in the ready-to-use composition. The thrombin preparation may contain other useful component, such as ions, buffers, excipients, stabilizers, etc. It is contemplated that the thrombin preparation contains human albumin, mannitol or mixtures thereof. Preferred salts are NaCl and/or CaCl₂, both used in the amounts and concentrations commonly applied for thrombin (e.g., 0.5 to 1.5% NaCl (e.g., 0.9%) and/or 20 to 80 mM CaCl₂ (e.g., 40 mM)).

Thrombin (or any other coagulation inducing agent, such as a snake venom, a platelet activator, a thrombin receptor activating peptide and a fibrinogen precipitating agent) can be derived from any preparation which is suitable for use in humans or animals (i.e., pharmaceutically acceptable). Suitable sources of thrombin include human or bovine blood, plasma or serum (thrombin of other animal sources can be applied if no adverse immune reactions am expected) and thrombin of recombinant origin (e.g., human recombinant thrombin); autologous human thrombin can be preferred for some applications.

The diluent preferably comprises a buffer or buffer system, for example at a pH of 3.0 to 10.0.

In various embodiments the present disclosure provides a hemostatic composition comprising the polysaccharide polymer, including amylopectin or dextrins contemplated herein in particulate form suitable for use in homeostasis (control of bleeding), wherein the composition is present in paste form containing a crosslinked biocompatible polymer in an amount of 5 to 30% (w/w), of 10 to 25% (w/w), or of 12 to 20% (w/w). In a further embodiment the crosslinked polymer, e.g., amylocpectin or a dextrin described herein, is present in an amount of 15.0 to 19.5% (w/w) (=weight of dry gelatin per weight of final composition), from 16.0 to 19.5% (w/w), from 16.5 to 19.5% (w/w), from 17.0 to 18.5% (w/w) or from 7.5 to 18.5% (w/w), from 16.5 to 19.0% (w/w) or 16.8 to 17.8% (w/w), or from 16.5 to 17.5% (w/w), and wherein the composition optionally comprises an extrusion enhancer, such as albumin. For example, if the extrusion enhancer is albumin (e.g., human serum albumin), it is provided in an amount of between 0.5 to 5.0% (w/w)(=weight of extrusion enhancer per weight of final composition), between 1.0 to 5.0% (w/w), between 2.0 to 4.5% (w/w), between 1.5 to 5.0% (w/w) or about 1.5% (w/w).

In various embodiments, the present disclosure relates to a hemostatic composition for use in the treatment of an injury selected from the group consisting of a wound, a hemorrhage, damaged tissue, bleeding tissue and/or bone defects.

In various embodiments the disclosure provides a method of treating an injury selected from the group consisting of a wound, a hemorrhage, damaged tissue and/or bleeding tissue comprising administering a hemostatic composition according to the present invention to the site of injury.

The present disclosure also provides a method for delivering a hemostatic composition according to the invention to a target site in a patient's body, said method comprising delivering a hemostatic composition to the target site. Although in certain embodiments, a dry composition can be directly applied to the target site (and, optionally, be contacted with the pharmaceutically acceptable diluent at the target site, if necessary), it is contemplated to contact the dry hemostatic composition with a pharmaceutically acceptable diluent before administration to the target site, so as to obtain a flowable hemostatic composition in a wetted form, especially a hydrogel form.

The diluents contemplated herein may further contain other ingredients, such as excipients. An “excipient” is an inert substance which is added to the solution, e.g., to ensure that thrombin retains its chemical stability and biological activity upon storage (or sterilization (e.g., by irradiation)), or for aesthetic reasons, e.g., color. Excipients include proteins, e.g., human albumin; carbohydrates, e.g., mannitol; polymers, e.g., polyethylene glycol (PEG); and sodium acetate. Concentrations of human albumin contemplated for the reconstituted product are from 0.1 to 100 mg/ml, or from 1 to 10 mg/ml. Mannitol concentrations can be in the concentration range of from 0.5 to 500 mg/ml, or from 10 to 50 mg/ml. PEG concentrations can be in the concentration range of from 0.5 to 500 mg/ml, or from 10 to 50 mg/ml. PEG average molecular weights may range from 500 to 20,000. Sodium acetate concentrations are in the range of from 1 to 10 mg/ml, or from 2 to 5 mg/ml.

In one embodiment, the final container further contains an amount of a stabilizer effective to inhibit modification of the polymer when exposed to the sterilizing radiation, preferably ascorbic acid, sodium ascorbate, other salts of ascorbic acid, or an antioxidant.

In various embodiments, a ready to use form of the present hemostatic composition may be provided with a diluent which can then be directly applied to the patient. Also contemplated is a method for administering or contacting a patient with a ready to use form of a hemostatic composition, wherein the hemostatic composition is provided in a first syringe and a diluent for reconstitution is provided in a second syringe, the first and the second syringe are connected to each other, and the fluid is brought into the first syringe to produce a flowable form of the hemostatic composition; and optionally returning the flowable form of the hemostatic composition to the second syringe at least once. This process (also referred to as “swooshing”) provides a suitable ready-to-use form of the compositions according to the present disclosure which can easily and efficiently be made also within short times, e.g., in emergency situations during surgery. This flowable form of the hemostatic composition provided herein is specifically suitable for use in the treatment of an injury selected from the group consisting of a wound, a hemorrhage, damaged tissue, bleeding tissue and/or bone defects. In various embodiments, the ready-to use preparations are present or provided as hydrogels.

For stability reasons, such products (as well as the products according to the present disclosure) are, in some embodiments, provided in a dry form in a final container and brought into the ready-to-use form (which is usually in the form of a hydrogel, suspension or solution) immediately before use, necessitating the addition of wetting or solvation (suspension) agents.

In various embodiments, a flowable form of the hemostatic composition contains particles of which more than 50% (w/w) have a size of 100 to 1000 μm, or particles of which more than 80% (w/w) have a size of 100 to 1000 μm. It is contemplated that the flowable form contains crosslinked amylopectin or dextrin in an amount of 5 to 30% (w/w), of 10 to 25% (w/w), or of 12 to 20% (w/w).

The hemostatic crosslinked polysaccharide polymer, including amylopectin or dextrin herein, according to the present disclosure—once applied to a wound—forms an efficient matrix which can form a barrier for blood flow. Specifically the swelling properties of the hemostatic polymer can make it an effective mechanical barrier against bleeding and re-bleeding processes.

Further components may be present in the hemostatic composition according to the present invention. In various embodiments, the hemostatic compositions according to the present invention may further comprise one or more of a substance selected from the group consisting of an antifibrinolytic, a procoagulant, a platelet activator, an antibiotic, a vasoconstrictor, a dye, a growth factor, bone morphogenetic proteins and pain killers.

The present disclosure also provides to a brushed final container. This finished container contains the hemostatic composition according to the present invention in a sterile, storage-stable and marketable form. The final container can be any container suitable for housing (and storing) pharmaceutically administrate compounds. Syringes, vials, tubes, etc. can be used; however, providing the hemostatic compositions according to the present invention in a syringe is preferred. Syringes have been a preferred administration means for hemostatic compositions as disclosed in the prior art also because of the handling advantages of syringes in medical practice. The compositions may then preferably be applied (after reconstitution if necessary) via specific needles of the syringe or via suitable catheters. The reconstituted hemostatic compositions (which are reconstituted to form a hydrogel) may also be applied by various other means, e.g., by a spatula, a brush, a spray, manually by pressure, or by any other conventional technique. Administration of the flowable hemostatic composition to a patient by endoscopic (laparoscopic) means is contemplated. In various embodiments, the hemostatic compositions are applied using a syringe or similar applicator capable of extruding the reconstituted composition through an orifice, aperture, needle, tube, or other passage to form a bead, layer, or similar portion of material. Mechanical disruption of the compositions can be performed by extrusion through an orifice in the syringe or other applicator, typically having a size in the range from 0.01 mm to 5.0 mm, preferably 0.5 mm to 2.5 mm.

Another aspect of the invention concerns a method for providing a ready-to-use hemostatic composition comprising contacting a hemostatic composition produced by the process according to the present invention with a pharmaceutically acceptable diluent.

Kits

Also contemplated herein are kits comprising a hemostatic composition useful to carry out the methods. The kit comprises a hemostatic composition according to the present disclosure and instructions for use.

It is contemplated that the kit further comprises, specifically if the hemostatic composition is contained in dry form, a pharmaceutically acceptable diluent for reconstitution of the hemostatic composition. Further components of the kit may be one or more devices for administration, such as syringes, catheters, brashes, etc, (if the compositions are not already provided in the device) or other components necessary for use in medical (surgical) practice, such as substitute needles or catheters, extra vials or further wound cover means. In various embodiments, the kit herein comprises a syringe housing a dry and stable hemostatic composition and a syringe containing the diluent (or provided to take up the diluent from another diluent container). It is further contemplated that the kit comprises interlocking syringes useful for mixing or “swooshing” the composition.

Additional aspects and details of the invention will be apparent from the following examples, which are intended to be illustrative rather than limiting.

### EXAMPLES

### Example 1—Preparation of Flowable Hemostatic Matrix

A series of cross-linked maltodextrins utlizing low (<2,000 Da), medium (20 kDa), and high molecular weight (>45,000 Da) maltodextrin (icodextrin) starting materials were prepared. Preparations varied in ratio of cross-linker (epichlorohydrin) and base relative to malotdextrin, reaction temperature, with or without the addition of surfactant, in aqueous or non-aqueous media, either stirred or under static conditions, and post synthesis processing (washing, grinding, drying). The hydrophilicity (swelling) of the hydrogels were assessed by measuring their water uptake (equilibrium swell) after incubating in 0.9% saline for 24 hours and its swell weight is determined. The sample is then dried at 120° C. for 2 hours and its dry weight is determined. The weight difference is used to calculate % equilibrium swell (E-Swell). Hydrogels prepared with icodextrin had E Swells in the range of 120 to 900% and Q Swells at 30 seconds in the range of 120 to 350. The reaction time was approximately 22 to 23 minutes. Hydrogels made with high molecular weight icodextrin showed E Swells in the range of 450 to 1200%. The reaction time was approximately 16 minutes.

Cross-linked maltodextrin 0092-120A was ground with a mortar and pestle and then sieved through a 0.5 mm sieve. The sieved material was evaluated as a flowable hemostatic matrix. A 5 mL syringe was loaded with 0.8 grams of the ground material and then connected to another syringe containing 3.8 mL of water. The syringes were swooshed back-and-forth about twenty times to hydrate the powder, and then left for about 30 seconds before extruding the hydrated material. Upon extrusion, a hydrogel with a consistency comparable to Floseal VH/SD hemostatic matrix was observed.

### Example 2—Methods to Assess Hemostatis Using the Hemostatic Composition

Hemostatic ability of the hemostatic cokpossition herein can be measured using several assays known in the art.

Thromboelastography (TEG) is performed as described in the art. For example, a TEG® 5000 Thromboelastography® Hemostasis System is used employing software TEG Analytical Software (TAS). In brief, 0.125 g of test article is reconstituted with 625 μl of the thrombin stock solution containing 500 IU/ml thrombin and 40 mMCaCl2 which is then left to sit for 5 min. Approximately 150 μl or 150 mg of the reconstituted test article is transferred to a TEG cup which is placed into the instrument. Immediately 210 μl of the blood anti-coagulated with 5 U/ml of heparin is added to the cup and quickly mixed. The TEG is then started and collects data for typically 20 minutes. The Amplitude (A) and Maximal Amplitude (MA) values are used to score product performance and compared to a reference standard. A and MA values >50 mm and an A/MA value of >1 are predictors of good hemostatic activity and robust clot formation.

Extrusion Force (EF) is measured as described in the art (see e.g., US 20130129710). EF analysis is performed to determine force values for 5 cc syringes with a male Luer lock system (with a cylindric body having an inner diameter of 0.482 inch) having a standard 6.35 cm delivery tip attached to it. In brief 0.80 g test article is transferred into a 5.0 ml Matrix (as described above) syringe. 4.0 ml Thrombin/CaCl₂ stock solution (containing 500 IU/ml thrombin and 40 mM CaCl2 and approx. 50 mg/ml albumin) is taken-up into a 5.0 ml standard syringe with a female luer lock system. The two syringes are connected and the test article rapidly reconstituted 20 times and then allowed to wait for 30±3 minutes prior to analysis. The interconnected syringes are then “swooshed” two more times, and the syringe with the male luer lock system containing the reconstituted sample is fitted with the applicator tip and inserted into the MTS INSIGHT™ Electromechanical force gauge. The sample is extruded at a set compression rate of 250 mm/minute, and its mean force determined over total sample extrusion is recorded.

A collagenase assay is useful to measure time to cell lysis which is an indication of residence time in vivo. For the assay, 0.08 g of the test and control samples are incubated with 2 ml of PBS puffer for 30 minutes at 37° C. in an end over end mixer. Thereafter the samples are subjected to centrifugation in an Eppendorf centrifuge at 14000 rpm for 5 minutes at room temperature. The supernatant is discarded and the precipitate re-suspended in 1.2 ml PBS buffer containing 0.111 U/ml collagenase. A reference sample is incubated with 1.2 ml PBS buffer (without the addition of collagenase). The samples are incubated at 37° C. in an end over end mixer and after defined standing times the supernatants are aspirated, weighed and collected for protein determination (BCA test) and samples are refilled with 1.2 ml of PBS buffer containing collagenase. The time to lysis can be determined by measuring the content of the degraded proteins that were released into the supernatants over time. Measuring the estimated 90% lysis time of the test article in this assay is an indirect estimate of the potential in vivo residence time of the hydrogel.

### Example 3—In-Vivo Assessment of Flowable Hemostatic Agents

Materials and Methods:

For this study, midline laparotomy is performed, followed by electrocautery to stop the bleeding from the surgical incision. The liver is exposed and a lobe is isolated. A 10 mm diameter punch biopsy is used to create a series of 2, non-full thickness lesions, approximately 5 mm deep, with the core tissue removed. A pre-treatment bleeding assessment is made on the lesion which includes collecting the blood flowing from each lesion for 6 seconds with pre-weighed gauze.

Test articles (i.e., flowable hemostatic compositions) were randomized and presented to the surgeon, who is blinded to the treatment prior to lesion creation. Approximately 1.0 ml of the assigned hemostatic test article was topically applied to a lesion. Saline moistened gauze is used to help approximate the test articles to their designated lesions, and the timer is started. The saline moistened approximation, gauze is removed after 2 minutes.

The degree of bleeding from each lesion is assessed immediately after gauze removal (time point 0) and at 2, 3, 5, 7 and 10 minutes thereafter, which is equal to 2, 4, 5, 7, 9, and 12 minutes after application.

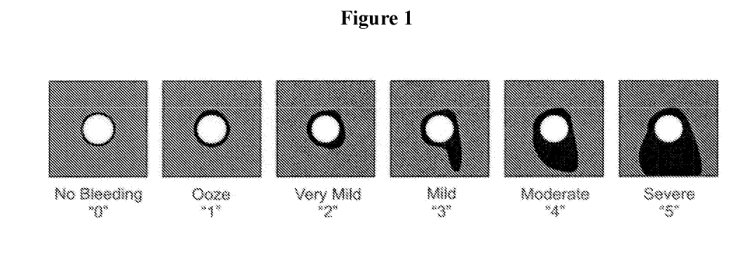

The qualitative post-treatment bleeding assessment was performed using a pre-defined scoring system (FIG. 1), wherein successful hemostasis is defined as a bleeding score of <1. Test items saturated with blood but without active bleeding receive a bleeding score of 0. Saline was used to irrigate the excess test articles away from the lesions after the 5 minute assessment. The procedure is repeated and performed in multiple liver lobes. A single surgeon creates, treats, and performs the observation assessments.

The test article powder was portioned in 5 mL syringes, (0.8 g/syringe) re-designated as Flowable Maltodextrin and evaluated in the porcine-liver punch model. The control composition used in was FLOSEAL VH S/D, 10 mL kit size. A solution containing 500 I.U./ml thrombin in 0.9% saline was prepared and was used to reconstitute each test article (3 mL/syringe).

Each test article sample was rapidly mixed by passage between syringes (“swooshed”) 20 times, and applied to lesions between 5 minutes-60 minutes after preparation. One milliliter aliquots of the reconstituted test article were dispensed into individual 3 mL volume syringes. These individual 3 mL syringes were then used to apply the test article to the liver punch lesions.

As a comparison, in-vivo evaluation of FLOSEAL VH S/D was also performed using the porcine liver punch-biopsy model. The compactor was prepared per the IFU and applied to lesions between 5-60 minutes after preparation. One milliliter aliquots of the reconstituted FLOSEAL Matrix were dispensed into individual 3 mL volume syringes. These individual 3 mL syringes were then used to apply the test article to the liver punch lesions at various time points.

Results:

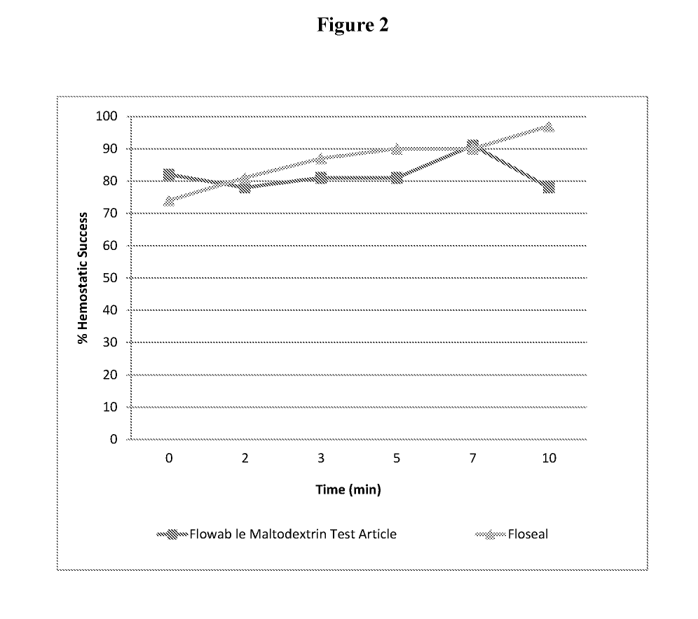

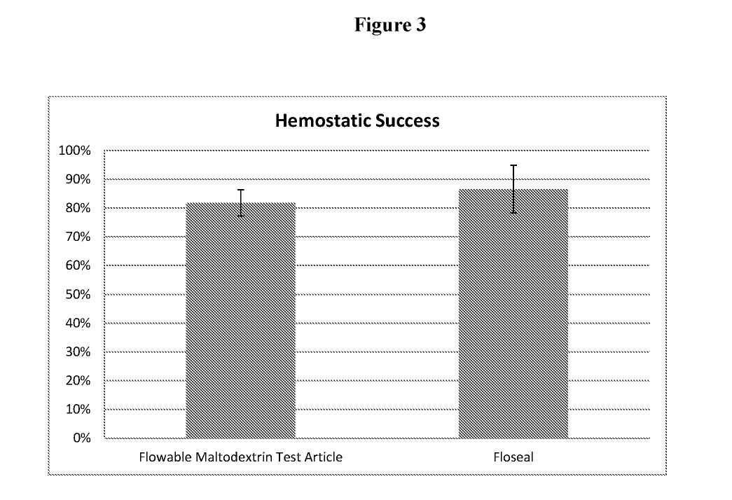

In-vivo evaluation of Flowable Maltodextrin and Floseal VH S/D was performed using the porcine liver punch-biopsy model in 4 animals, 1 animal per day. The results are presented in Table 1 and Figures. 2 and 3. The pre-application bleeding rates for the two sample groups (Maltodextrin Flowable and FLOSEAL VH S/D) are presented in Table 1. Pre-treatment bleeding rates were similar across treatment groups indicating FLOSEAL and Sierra Flowable were administered to comparable liver lesions.

TABLE 1
Summary Pre-Application Bleeding Rates (mL/min).

| | Maltodextrin Flowable | FLOSEAL VH S/D |
|---|---|---|
| Mean | 6.965 | 7.187 |
| N | 32 | 31 |
| SD | 3.847 | 4.420 |
| Minimum | 0.947 | 2.172 |
| Median | 6.384 | 6.666 |
| Maximum | 14.324 | 20.056 |

The data presented in FIG. 2 demonstrate hemostatic success at various time points during the hemostatic evaluation in 78.1-90.6% of lesions treated with Flowable Maltodextrin (74.2-96.8% for Floseal VH S/D). Across all lesions, Flowable Maltodextrin treatment led to hemostatic success in 157 out of 192 timepoints (81.8±4.6%) compared to 161 out of 186 timepoints (86.6±8.0%) treated with Floseal VH S/D (FIG. 3). Flowable maltodextrin prepared as per Example 1, led to hemostatic success and reduction in bleeding generally comparable to that of FLOSEAL VH S/D.

Numerous modifications and variations of the invention as set forth in the above illustrative examples are expected to occur to those skilled in the art. Consequently only such limitations as appear in the appended claims should be placed on the invention.

1. A hemostatic composition comprising a cross-linked amylopectin, icodextrin or maltodextrin or a cross-linked modified amylopectin, icodextrin or maltodextrin, wherein the composition is a flowable hydrogel, the hemostatic composition further comprising a procoagulant.

2. The hemostatic composition of claim 1, further comprising one or more agents selected from the group consisting of a blood clotting factor, a styptic, an astringent, and other hemostatic agent.

3. The method of claim 1, wherein the procoagulant is thrombin.

4. The hemostatic composition of claim 1, wherein amylopectin, icodextrin or maltodextrin further comprises a functional group selected from the group consisting of an amine, an aldehyde, a phosphate, a phosphonate, a sulfate, a sulfonate, and a carboxylate group.

5. The hemostatic composition of claim 1, wherein the composition activates the coagulation cascade or enhances binding to tissues.

6. The hemostatic composition of claim 1, wherein the cross-linked amylopectin, icodextrin or maltodextrin or cross-linked modified amylopectin, icodextrin or maltodextrin comprises a crosslinking agent selected from the group consisting of epichlorohydrin, bis-epoxypropylether, ethylene glycol-bis-epoxy propyl ether, sodium trimetaphosphate (STMP), adipic-acetic anhydride, phosphorous oxychloride, formaldehyde, a diepoxide, vinylcyclohexene dioxide, butadiene dioxide, formaldehyde, gluaraldehyde and genipin.

7. The hemostatic composition of claim 1, wherein the maltodextrin is selected from the group consisting of low molecular weight, medium molecular weight or high molecular weight maltodextrin.

8. The hemostatic composition of claim 7, wherein the low molecular weight maltodextrin is less than 2,000 Da.

9. The hemostatic composition of claim 7, wherein the medium molecular weight maltodextrin is between 2,000 and 45,000 Da.

10. The hemostatic composition of claim 7, wherein the high molecular weight maltodextrin is greater than 45,000 Da.

11. The hemostatic composition of claim 1, further comprising one or more of an additive or active agent selected from the group consisting of a polysaccharide, polyvinylalchohol, polyethyleneimine, chitosan, alginic acid, a fucoidan, hyaluronic acid, heparin, heparan, chondroitin sulfate, amylopectin, amylose, xanthan gum, guar gum, carageenans, carboxymethyl cellulose, carboxymethyl maltodextrin, carboxymethyl icodextrin, carboxymethyl starch, agarose, and natural or synthetic polymers.

12. The hemostatic composition of claim 1, wherein the amylopectin, icodextrin or maltodextrin is derived from plant material, bacteria, fungi or algae.

13. The hemostatic composition of claim 12, wherein plant material is selected from the group consisting of corn, soy, rice, tapioca, wheat, waxy corn and waxy rice.

14. A method of treating an injury selected from the group consisting of a wound, a hemorrhage, damaged tissue and bleeding tissue comprising administering a hemostatic composition according to claim 1 to the site of injury.

15. A method for slowing or stopping bleeding, comprising applying to a bleed area a hemostatic composition according to claim 1.

16. The method of claim 14, wherein the injury or bleeding occurs during surgery.

17. The method of claim 14, wherein the composition is used in a sponge, pad, film or fiber.

18. The method of claim 15, wherein the composition stops bleeding within seconds or minutes.

19. A kit for the treatment of an injury selected from the group consisting of a wound, a hemorrhage, damaged tissue and bleeding tissue or for slowing or stopping bleeding comprising a) a hemostatic composition according to claim 1 and b) instructions for use.
